(19) United States
(12) Patent Application Publication
(10) Pub. No.: US 2004/0199186 A1
(43) Pub. Date: Oct. 7, 2004

(54) Implant to promote axon regeneration across spinal cord and peripheral nerve gaps

(76) Inventors: Suzanne Elizabeth Kuffler, Woods Hole, MA (US); Ivan Sosa, San Juan, PR (US); Onix Reyes, Guynabo, PR (US)

Correspondence Address: Damien Kuffler, 201 Blvd. del Valle, San Juan, PR, 00901, US

(21) Appl. No.: 10/406,330

(22) Filed: Apr. 4, 2003

(51) Int. Cl. A61B 17/03

(52) U.S. Cl. 606/152

(57) ABSTRACT

Presently there are no means to make nerves regenerate across a gap caused by an injury to the spinal cord or peripheral nerves that will lead to neurological recovery. This invention involves the use of a biocompatible and bioresorbable tube that is placed in the gap. The tube is filled with a biocompatible and bioresorbable 3-dimensional matrix. The matrix is a material that induces nerves to regenerate. To improve the potency of the matrix to promote nerve regeneration a combination of factors is infused into the matrix The factors are infused by means of a catheter attached to a subcutaneous pump with its tip placed halfway between the ends of the tube with its other end. This unique device will induce neurological recovery that is presently not possible.

### CROSS-REFERENCE TO RELATED APPLICATIONS

Non Applicable

### STATEMENT REGARDING FEDERALLY SPONSORED RESEARCH OR DEVELOPMENT

Non Applicable

### REFERENCE TO A MICROFICHE APPLENDIX

Non Applicable

### BACKGROUND OF THE INVENTION

### FIELD OF INVENTION

Repair of the Spinal Cord and Peripheral Nerves

Trauma to the human spinal cord and peripheral nerves typically cuts the axons (nerves) and damages the nervous tissue leading to the formation of scar tissue or a gap in the spinal cord or peripheral nerve. Recovery of neurological function following nerve trauma requires surgical intervention. This intervention involves removing the damaged nervous tissue and then attempting to induce the injured axons to regenerate across the gap in their pathway and into the other end of the nerve or spinal cord. Peripheral axons can be induced to grow only across short but not long gaps (typically 1.5 to 2 cm). In the case of the human spinal cord injury the axons never regenerate and it has never been possible to promote any recovery of neurological function.

### STATE OF TECHNOLOGY

To allow damaged peripheral axons to regenerate across a peripheral nerve gap longer than 3 mm requires inserting a conduit between the cut ends of the peripheral nerve. But, an empty conduit promotes very limited axon outgrowth. The number of axons that regenerate across the gap can be increased if the conduit is filled with a 3-dimentional matrix that is permissive to axon regeneration. However, even such a matrix-filled conduit only allows axons to regenerate across relatively short gaps, rarely longer than 2 cm. Further, the extent of neurological recovery is typically minimal because only a small number of axons regenerate through such conduits. To induce larger numbers of axons to regeneration across long gaps requires the environment within the conduit be enriched with factors that promote axon regeneration.

Following a spinal cord lesion no neurological recovery has ever been reported. The first step for inducing neurological recovery is to induce the spinal cord axons to regenerate across the gap caused by the trauma. However, once the axons reach the spinal cord they can not regenerate into the cord because the spinal cord itself is inhibitory to axon in-growth. To promote axons to regenerate into the spinal cord requires factors that neutralize the regeneration-inhibiting molecules and thereby change the cellular environment of the spinal cord from one that inhibits regeneration to one that is permissive to and promotes regeneration.

No devices have been developed containing a 3-dimensional matrix plus the necessary regeneration-inducing factors to induce peripheral axons to regenerate across a long gap. Similarly, no devices have been developed that induce the regeneration of spinal cord axons across a gap and to change the spinal cord to being regeneration permissive.

In the United States alone there are 10,000 new quadriplegics and paraplegics annually. Presently there is absolutely no hope of any neurological recovery for these individuals. Further, each year in the United States 100s of thousands of individuals suffer permanent neurological deficits due to peripheral nerve and spinal cord injuries that can not be repaired. Therefore, it is essential to develop and use novel techniques to induce nerve regeneration following spinal cord and peripheral nerve injuries that lead to neurological recovery.

In conclusion, although a person with a long peripheral nerve gap might recover limited neurological function, in most cases there is no neurological recovery. Presently a person with a spinal cord lesion will forever suffer complete neurological losses. To make matters worse, a significant number of these individuals suffer due to the development of chronic pain that occurs as a direct result of their injuries. Some secondary effects of the failure of neurological recovery are severe degenerative changes in joints, physiological deficits, and organ failure.

### WHY IS THIS DEVICE AN INNOVATION

Peripheral axons have the ability to regenerate after an injury and there is a great deal of data about various factors that induce axon regeneration. However, there has been little success in inducing peripheral nerve axons to regenerate across a gap longer than 2 cm. Thus, most patients with an injury resulting in a long peripheral nerve gap suffer permanent neurological losses.

In the case of a lesioned human peripheral nerve resulting in a gap the standard surgical technique involves suturing a length of donor nerve from the patient across the nerve gap. However, these nerve bridges induce only a limited number of axons to regenerate across the bridge and neurological recovery is limited.

Due to the enormous complexity of the human spinal cord it has been extremely difficult to determine why injured human spinal cord axons do not regenerate and how to induce them to regenerate. As a result, all individuals who suffer a spinal cord lesion suffer permanent neurological losses.

Various animal models have been used to test methods for promoting axons to regenerate across a spinal cord gap and into and within the other end of the spinal cord. Some methods have induced axon regeneration and neurological recovery, but at best the number of axons that regenerate and the neurological recovery is minimal (<10%). Further, the techniques do not provide consistent results. Thus, even for animal models no methods or devices have been developed that promote significant axon regeneration and neurological recovery.

### BRIEF SUMMARY OF INVENTION

The invention consists of a biocompatible tube just larger than the diameter of the peripheral nerve or spinal cord to be repaired. The tube is filled with a porous biocompatible 3-dimensional matrix. The matrix-filled tube is placed in the gap between the cut ends of a spinal cord or peripheral nerve. The ends of the peripheral nerve or spinal cord are inserted 3 millimeters into the ends of the tube and sutured in place. One end of a catheter is attached to a pump while the other end is inserted inside the tube. The catheter allows the infusion into the matrix of factors that promote axon regeneration and other factors that block the action molecules that inhibit axon regeneration.

Although conduits have been used to repair severed peripheral nerves, they have not been used together with infused factors. Therefore, the effectiveness of such devices in inducing axon regeneration is restricted to promoting regeneration across only short peripheral nerve gaps.

The present invention has significant improvements over all previously used conduits. (1) The invention provides a consistent and reproducible cellular environment across a gap in a human spinal cord or peripheral nerve. (2) The invention uses the infusion of a combination of factors that promote axon regeneration, as well as factors that neutralize molecules that inhibit axon regeneration, that have never before been used. (3) The combination of factors to be infused can be changed, or their rations modified as required by changing the factors in the pump reservoir. (4) The concentration of factors infused into the 3-dimentional matrix can be controlled by altering the rate at which they are infused. Thus, this invention will promote axons to regenerate across long spinal cord and peripheral nerve gaps leading to a high rate of neurological recovery.

### BRIEF DESCRIPTION OF THE DRAWING

FIG. 1 of 1.

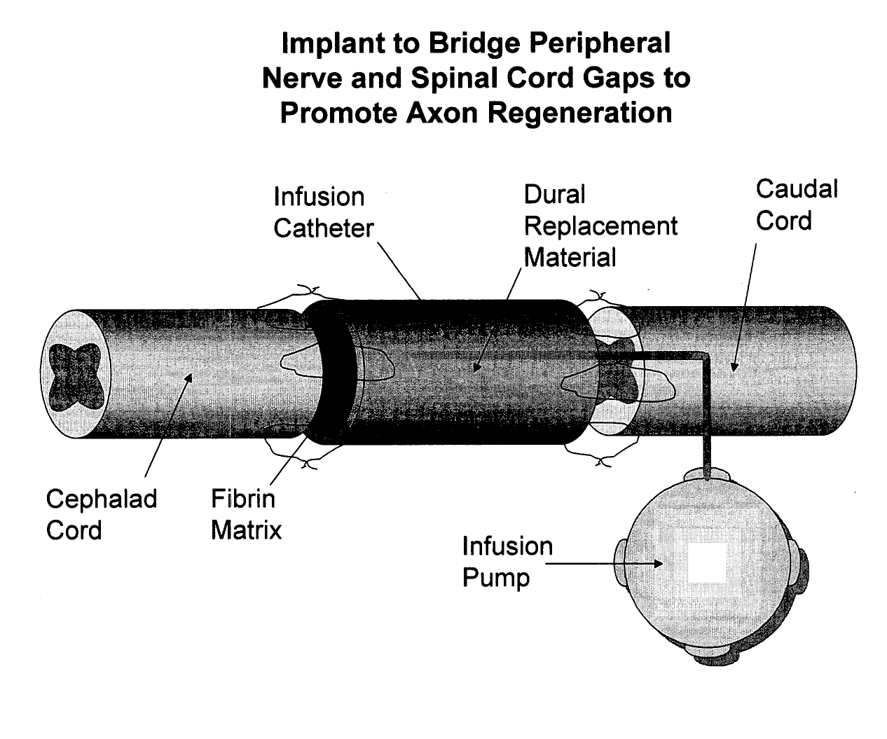

The device consists of a biocompatible tube filled with a biocompatible 3-dimensional matrix. The tube to be inserted must be just larger than the diameter of the spinal cord or peripheral nerve to be repaired. The device is placed within a gap of a sectioned spinal cord or peripheral nerve. The ends of the spinal cord/peripheral nerve are inserted about 3 mm inside the tube. The ends within the tube are secured with sutures through the tube and the perineurium of the peripheral nerve, or the dura of the spinal cord.

A catheter is inserted along the inside wall of the tube with its end positioned at the middle of the tube in the case of repair of a spinal cord or at the distal end in the case of peripheral nerve repair. The other end of the tube is attached to a battery-powered pump. The pump will be placed in a subcutaneous pouch of the patient. The pump can be turned on and off, as well as its pump rate controlled, by means of an external controller that operates trans-cutaneously.

### DETAILED DESCRIPTION OF THE INVENTION

Following a complete anatomical lesion of the human spinal cord or peripheral nerve the damaged nervous tissue must be removed up to the level of the healthy cephalad and caudal ends of the spinal cord or peripheral nerves. This is done to prevent tissue scarring that would prevent axon regeneration. Although the surgery leaves a gap between the cephalad and caudal ends of the spinal cord and peripheral nerve it does not cause neurological deficits in addition to those caused by the initial trauma.

The invention consists of a biocompatible tube filled with a biocompatible 3-dimensional matrix. The tube serves as a bridge between the cut ends of the spinal cord or peripheral nerve and provides mechanical stability within the gap. The tube is filled with a 3-dimensional matrix that is injected into the tube. Regenerating axons grow through the matrix within the tube to its other end where they regenerate into the distal portion of the peripheral nerve, or into both the cephalid and caudal ends of the spinal cord leading to reinnervation of the original nerve targets and functional neurological recovery.

The tube to be implanted in a patient must have an internal diameter slightly larger than the diameter of the thecal sack of the cephalad and caudal ends of the sectioned spinal cord, and be just larger than the diameter of the peripheral nerve to be bridged. The diameter of the spinal cord varies from the cephalad to the caudal end, as well as between children and adults. Similarly there is a large variation in the diameters of peripheral nerves. Thus, tubes will be manufactured in a range of sizes suitable for implantation into a spinal cord and peripheral nerve gap of a young child to an adult.

The tube to be implanted will be selected at the time of surgery to have an internal diameter appropriate for the nervous tissue to be repaired. The tubes will be manufactured longer than the length of the gaps to be bridged by the tube. This allows the selected tube to be cut to the precise length needed just before it is implanted (6 mm longer than the gap to be bridged). This length allows the ends of the peripheral nerves and spinal cord to be inserted 3 mm into either end of the tube.

Trauma to the spinal cord frequently damages the dura that surrounds the cord and the dura must be repaired to prevent leak of cerebral spinal fluid in which the spinal cord is bathed. The tube used to bridge a spinal cord gap will serve as a replacement of the damaged dura membrane. The interior surface of the tube provides a substrate onto which a natural dura sac can attach as it develops to reestablished a continuous dura between the cephalad and caudal portions of the sectioned spinal cord. The tube also protects the gap from the invasion of cells that might induce scarring and prevent axon regeneration. For both the spinal cord and peripheral nerve the tube helps direct the growth of the axons across the gap.

The selected tube will be filled just before use with a porous biocompatible and biodegradable 3-dimensional matrix. The matrix material has been shown to promote axon regeneration. The matrix provides a 3-dimensional scaffold through which the regenerating axons can grow entirely across the gap to where they can find their appropriate target nerve tracts into which they will continue to grow. Because the matrix contains an inherent number of growth factors it serves as a potent promoter of nerve regeneration. The matrix also allows additional growth and other factors to be infused into it and through which they diffuse creating a concentration gradient of the factors.

The tube containing the 3-dimensional matrix will be placed in the gap in the spinal cord or peripheral nerve. The ends of a sectioned spinal cord or peripheral nerve will be inserted 3 millimeters into the ends of the tube and into contact with the matrix inside the tube. The ends of the tube will be sutured in place through the wall of the tube and the dura surrounding the ends of the sectioned spinal cord and to the perineurium of the peripheral nerves.

Although the matrix in the tube promotes axon regeneration, the number of axons that regenerate into the gap and the distance they regenerate, can be significantly increased by infusing the matrix with a cocktail of neurotrophic and other factors. Infusion of these factors will induce a larger number of axons to regenerate completely across the gap and to the ends of the spinal cord or peripheral nerve. The combination of factors is one we have demonstrated to promote regeneration of both sensory and motor axons. In the case of spinal cord gaps the matrix will also be infused with factors that neutralize molecules that inhibit axon regeneration. Infusion of these molecules will change the cellular environment of the spinal cord from one that inhibits regeneration to one that is permissive to and promotes axon regeneration.

A catheter will be attached to a pump that is implanted sub-cutaneously. The other end of the catheter will be inserted along the inside wall of the tube and secured in place with a suture around the catheter and through the wall of the end of the tube. The blind-ended catheter has 6 holes along its last few millimeters and through which the factors will be infused into the matrix. The pump reservoir can be filled prior to implantation and can be refilled by trans-cutaneous injection into its reservoir as needed.

Damaged spinal cord axons must regenerate in both the cephalic and caudal directions. Therefore, to induce regeneration of spinal cord axons in both directions the end of the catheter will be positioned at the middle of the spinal cord gap. This position pumps the factors into the center of the 3-dimensional bridging the gap from where they diffuse laterally. Lateral diffusion of the factors leads to the formation of concentration gradients of the factors in both directions through matrix. The concentration gradients are highest at the middle of the gap and lowest at the cephalad and caudal ends of the gap. We have shown that such concentration gradients promote and direct axon regeneration up the concentration gradients. Axons regenerating in both the cephalad and caudal directions will be equally induced to regenerate up the gradients and towards the center of the spinal cord gap. Once the axons reach the center of the spinal cord gap they will no longer required the concentration gradient of pumped factors because they will continue to grow towards their respective ends of the spinal cord due to signals released from cells in those ends. Thus, cephalad motor axons and caudal sensory axons are equally induced to regenerate leading neurological recovery of both sensory and motor function.

Injured peripheral axons only regenerate in the distal direction. Therefore, to repair a peripheral nerve the end of the catheter will be inserted centrally from the caudal end of the tube about 3 mm into the matrix. The factors pumped into the matrix will diffuse along the matrix and create a concentration gradient of the infused factors from the caudal to the central end of the nerve gap. This will induce the axons to grow up the concentration gradient of the factors, across the entire length of the gap and into the distal nerve. From here they will regenerate along the distal portion of the nerve to their targets which they will innervate.

The tubes will be manufactured, packaged and gamma irradiated to sterilize the material. Packaged sterile tubes will be available of varying diameters and lengths to suite all anticipated needs. Thus the tubes will be available to surgeons for selecting the tube of with the appropriate diameter and length at the time when this information is determined, which is when the tube must be implanted in a patient.

The present invention will provide a consistent and reproducible cellular environment across a gap in the human spinal cord or peripheral nerve. The invention will also provide the infusion of a combination of factors that promote axon regeneration and factors that neutralize molecules that inhibit axon regeneration. No devices have been developed that infuse such factors or combination of factors to induce axon regeneration across a gap of any length in a spinal cord or peripheral nerve leading to neurological recovery. This device will promote axons to regenerate across spinal cord and peripheral nerve gaps leading to neurological recovery.

Invention

Why is the development of this device non-obvious?

The complexity of the human spinal cord is enormous. Although much is known about how it functions when intact, virtually nothing is known about how to repair the human central nervous system after it has been injured. We are virtually the only research group in the world working full time with isolated adult human neurons. These neurons are removed from adult human organ donors. These tissues have allowed us to develop the only methods by which adult human neurons can be isolated and maintained alive and physiologically functional in the laboratory for long periods of time (more than 2 months). Although other laboratories carry out experiments with adult human neurons they are able to maintain the neurons viable for only up to only 24 hours. Our experience has given us the unique opportunity to study these neurons in ways that are not possible for other laboratories.

Many laboratories are studying how to repair peripheral nerves in animal models. However, most of these laboratories are only looking at the influences of one or two factors acting simultaneously to promote axon regeneration. Our group is one of the few working with complex combinations of physiologically critical factors to promote axon regeneration. We have found that these complex combinations exert almost 10 times the influence on nerve regeneration as one or 2 factors. We have carried out these experiments using adult human peripheral nerves as well as on animal peripheral nerve models.

Our experiments have allowed us to study: (1) the influences of various 3-dimensional matrices on axon outgrowth from adult human neurons; (2) mechanisms by which to turn off the influence of specific factors that inhibit axon regeneration from these neurons; (3) to develop complex combinations of factors that promote optimal axon outgrowth; and (4) to develop methods to direct the growth of regenerating axons up concentration gradients of the combined factors. However, it was only by combining these techniques that we developed a device that both promotes and directs a significant number of human neurons to extend processes that are many centimeters long. Without our extensive experimental experience with adult human neurons, experiments on the lesioned lesioned peripheral nerves, and our development of a number of different techniques, the development and construction of this device would have been non-obvious.

The pump and catheter to be used in conjunction with our device are patented by Medtronics Corp., and are FDA approved for the routine delivery of anti-inflammatory drugs and analgesics. No drugs delivered in this manner have induced axon regeneration. Thus, the standard uses of the pump and catheter have nothing to do with the promoting the repair of the injured human spinal cord.

The pump and catheter will be used in conjunction with our device to deliver factors that create a cellular environment that promotes axon regeneration across the spinal cord and peripheral nerve gap and that allow axon regeneration into the opposite ends of the spinal cord and peripheral nerve. Thus, using of the pump and catheter in conjunction with our device will vastly expand the effective uses of the pumps and catheters.

1. This device is an improvement on previous devices used to try and repair peripheral nerves and the spinal cord.
1. Previous devices have been composed of a simple non-resorbable empty tube.
2. Wherein this improvement comprises a bio-resorbable tube, a nerve regeneration-promoting 3-dimensional bio-resorbable matrix that completely fills the tube, and a device inserted within the 3-dimensional matrix with which to infuse the matrix with factors that will promote nerve regeneration.
3. This unique use of a bio-resorbable tube, a bio-resorbable matrix, and device to infuse factors into the matrix comprises an significant improvement over previously used devices and a completely new relationship of components that have never before used together.
We claim that this is a unique device comprises a major improvement over all previous devices designed to bridge a gap in the nervous system and that the use of bio-resorbable materials in the form of a tube filled with a 3-dimensional matrix infused with factors will lead to the significant repair of severed nerves and the spinal cord that has never before been achieved by any other device.
